(19) United States
(12) Patent Application Publication
(10) Pub. No.: US 2019/0058351 A1
(43) Pub. Date: Feb. 21, 2019

(54) INFORMATION DISPLAY DEVICE

(71) Applicant: YongLin Biotech Corp., New Taipei (TW)

(72) Inventors: PENG CHANG, New Taipei (TW); JEN FANG CHENG, New Taipei (TW)

(21) Appl. No.: 16/107,176

(22) Filed: Aug. 21, 2018

(30) Foreign Application Priority Data
Aug. 21, 2017 (CN) 201710718096. 9

(51) Int. Cl. H02J 7/34; G06F 1/16; G09G 3/34; G16H 10/60

(52) U.S. Cl. CPC H02J 7/345; G06F 1/1635; G09G 2330/04; G16H 10/60; G09G 2330/021; G09G 3/344

(57) ABSTRACT

An information display device adaptable in a ward to display patient information, which comprises: a control circuit; an electronic paper (epaper) module configured to be controlled by the control circuit for displaying patient information; a solar charging panel configured to output a first voltage; a battery configured to output a second voltage; a rechargeable energy storage device, and a voltage regulating circuit coupled to the solar charging panel, the battery, and the rechargeable energy storage device and configured to provide an operational voltage to the control circuit and the epaper module.

### CROSS-REFERENCE TO RELATED APPLICATIONS

This application claims priority to China Patent Application No. CN201710718096.9, filed on Aug. 21, 2017, the contents of which are incorporated by reference herein.

### FIELD

The present disclosure pertains to an information display device, and more particularly, to an information display device that can be installed in a ward to display patient information.

### BACKGROUND

In the ward of a general hospital, for the convenient display of a patient's basic information (e.g., name, type of illness, hospitalization status, etc) for the patents, family members, or medical staffs, bedside cards with hand written or printed information are commonly placed on the patient's beds.

With the advancement of electronic technology, bedside cards have gradually evolved into electronic display devices on which data can be updated by computers or servers. Such electronic bedside cards require stable power input to operate properly. Also, the design of such electronic information display device are required to minimize interference to the patient as well as reducing work load for the medical staff

### SUMMARY

One aspect of the instant disclosure provides an information display device adaptable in a ward to display patient information, which comprises a control circuit, an electronic paper (epaper) module, a solar charging panel, a battery, a rechargeable energy storage device, and a voltage regulating circuit. The electronic paper (epaper) module is configured to be controlled by the control circuit for displaying patient information. The solar charging panel is configured to output a first voltage. The battery is configured to output a second voltage. The voltage regulating circuit is coupled to the solar charging panel, the battery, and the rechargeable energy storage device, and is configured to provide an operational voltage to the control circuit and the epaper module. The rechargeable energy storage device may include one or more supercapacitor.

In some embodiments, the operational voltage includes a lower limit value and an upper limit value. The lower limit value of the operational voltage is higher than the lowest operating voltage of either one of the control circuit and the epaper module. One the other hand, the upper limit value thereof is lower than highest operating voltage of either one of the control circuit and the epaper module.

In some embodiments, when the battery is electrically connected to the voltage regulating circuit, if the voltage regulating circuit detects that a voltage of the battery is higher than an operating voltage on a main line, the voltage regulating circuit enables the batter to charge the rechargeable energy storage device. If the rechargeable energy storage device is fully charged, and the voltage of the battery is still higher than the operating voltage on the main line, the voltage regulating circuit discharges the battery until the voltage of the battery substantially equals to the operating voltage of the battery.

In some embodiments, the control circuit is configured to be operable in a working mode and a sleep mode. Upon awaken, the control circuit is configured to wirelessly connect to a server through a predetermined network connection setting, obtain a display data, cause a display refresh on the epaper module, and enter sleep mode after the display refresh.

In some embodiments, the control circuit includes a first circuit and a second circuit. The second circuit may include a timer. The timer may be configured to wake up the first circuit at a predetermined periodic time interval. The first circuit is configured to wirelessly connect to a server through a predetermined network connection setting for receiving a data.

Embodiments in accordance with the instant disclosure may efficiently prolong the operating duration of the information display device through the power from the solar charging panel, thus reducing the battery swapping frequency, thereby increasing operational convenience.

Another aspect of the instant disclosure provides an information display device, which comprises a control circuit, an epaper module, a Universal Serial Bus (USB) connector, a solar charging panel, a battery, a rechargeable energy storage device, and a voltage regulating circuit. The epaper module is configured to be controlled by the control circuit for displaying patient information. Only aVCC pin of the USB connector is coupled to a constant current circuit. The GND pin of the USB connector is coupled to ground. The voltage regulating circuit is coupled to the solar charging panel, the constant current circuit, the battery, and the rechargeable energy storage device, and configured to provide the control circuit and the epaper module an operational voltage. The constant current circuit is configured to provide power to the voltage regulating circuit only when the USB connector is coupled to an external device.

A further aspect of the instant disclosure provides an information display device adaptable in a ward to display patient information, which comprises: a solar module; a battery; a supercapacitor module; a voltage regulating circuit coupled to the solar module, the batter, and the supercapacitor module; an electronic paper (epaper) module; and a controller, configured to control the epaper module for displaying patient information, wherein the voltage regulating circuit selectively charges the supercapacitor module using the solar module and the battery. The epaper module and the controller are primarily powered by the supercapacitor module.

In some embodiments, the solar module further comprises a solar panel and a second supercapacitor. When the controller and the epaper module are in sleep mode, the second supercapacitor is configured to store energy generated by the solar panel, and upon awaken of the controller, to provide power thereto.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 shows a functional block diagram of an embodiment of an information display device according to the present disclosure.

FIG. 2 shows a functional block diagram of an embodiment of an information display device according to the present disclosure.

FIG. 3 shows a functional block diagram of another embodiment of the information display device according to the present disclosure.

FIG. 4 shows a functional block diagram of another embodiment of the information display device according to the present disclosure.

FIG. 5 shows an exemplary operational waveform diagram.

FIG. 6 shows a functional block diagram of another embodiment of the information display device according to the present disclosure.

FIG. 7 shows a functional block diagram of another embodiment of the information display device according to the present disclosure.

FIG. 8 shows an operation flow diagram of the control circuit unit of an information display device in accordance with one embodiment of the present disclosure.

FIG. 9 shows a schematic diagram of an exemplary example of the operation of FIG. 8.

### DETAILED DESCRIPTION

Embodiments of the instant disclosure will be specifically described below with reference to the accompanying drawings.

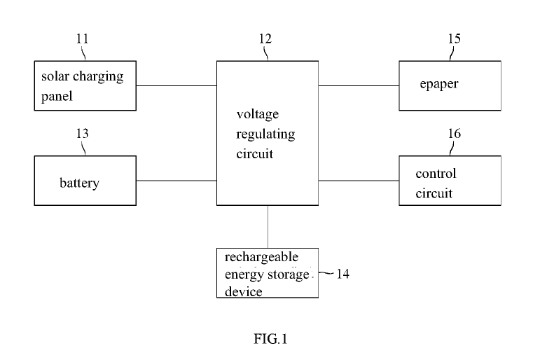

FIG. 1 shows a functional block diagram of an embodiment of an information display device according to the present disclosure. The information display device includes a solar charging panel 11, a voltage regulating circuit 12, a battery 13, a rechargeable energy storage device 14, an electronic paper 15, and a control circuit 16. The control circuit 16 is configured to transmit display data to the electronic paper 15 for generating image. The control circuit 16 further includes a wireless communication module (not shown) for connecting to a server to obtain related information, and is controlled by the control circuit 16 to convert the related information into display material. In one embodiment, the rechargeable energy storage device 14 includes one or more supercapacitor EDLC) arranged in parallel configuration.

In one embodiment, the information display device can be applied to an electronic bedside card in a ward for displaying patient related information, such as the name of the patient, the attending physician, the nurse on duty, the patient's special medical history, and the like. In another embodiment, the information display device is a patient physiological information recording device placed beside a patient's bed. Accordingly, the related information may include the patient's body temperature, blood pressure, pulse, and the like. In another embodiment, the information displayed by the display device can be directly input by a caregiver through the server or an authenticated handheld electronic device, and the control circuit 16 may control the display of image information on the epaper 15.

The voltage regulating circuit 12 has a first power input node coupled to the solar charging board 11 and a second power input node coupled to the battery 13, and a third power input node coupled to the rechargeable energy storage device 14. The voltage regulating circuit 12 further includes a power output node coupled to the electronic paper 15 and the control circuit 16. In this embodiment, the operating voltage range of the electronic paper 15 is 2.7V˜3.6V, and the operating voltage range of the control circuit 16 is 2.1˜V˜3.8V. The voltage regulating circuit 12 controls the voltage of the output node and maintains the voltage range substantially between 2.1V to 3.8V, so as to ensure proper operation of the electric paper 15 and the control circuit 16. In other words, one function of the voltage regulating circuit 12 is to ensure that the electronic paper 15 and the control circuit 16 can receive correct operating voltage. Further, another function of the voltage regulating circuit 12 is to serve as a protection circuit for the electronic paper 15 and the control circuit 16, so that the electronic paper 15 and the control circuit 16 will not be damaged upon the receipt of an excessive voltage.

In one embodiment, the voltage of the battery 13 (e.g., 4.2V) is higher than an upper regulating range limit of the voltage regulating circuit 12 (e.g., 3.6V). If the battery 13 directly supplies voltage to the electronic paper 15 or the control circuit 16, the electronic paper 15 or the control circuit 16 may be damaged. Therefore, in one embodiment, when the battery 13 is electrically connected to the voltage regulating circuit 12, the voltage regulating circuit 12 will first discharge the battery 13. The voltage regulating circuit 12 is configured to enable power supply from the battery 13 to the electronic paper 15 and the control circuit 16 when the voltage of the battery 13 would not cause damage to the electronic paper 15 or the control circuit 16. In another embodiment, the voltage regulating circuit 12 is configured to first allow the battery 13 to charge the rechargeable energy storage device 14; and when the rechargeable energy storage device 14 is fully charged, then allows the discharge or power supply to the electronic paper 15 and controls circuit 16. If the electronic paper 15 and the control circuit 16 is not in operation or enter into a sleep mode while the rechargeable energy storage device 14 has been fully charged, the battery 13 will continue discharge through the voltage regulating circuit 12 until the voltage of the battery 13 and that of the rechargeable energy storage 14 reaches equilibrium.

The solar charging panel 11 is configured to charge the rechargeable enemy storage device 14 through the voltage regulating circuit 12. The charging voltage of the solar charging panel 11 is higher than the upper regulation range limit of the voltage regulating circuit 12 (e.g., 3.6V). The saturation voltage of the rechargeable energy storage device 14 is configured to be greater than the charging voltage provided by the solar charging panel 11, so as to protect the rechargeable energy storage device 14. In one embodiment, the upper tolerable voltage limit of the rechargeable energy storage device 14 is 5.5V.

Electronic paper is known for low power consumption, and the original picture and text can be displayed even in the absence of power. Nevertheless, the power consumed by the control circuit 16 may still shorten the operable duration of the information display device. In the present disclosure, the information display device may generate power through the solar charging panel 11 and stores it in the rechargeable energy storage device 14. And through the power consumption calculation and circuit design, the power generated by the solar charging panel 11 may be equal to or greater than the daily power consumption of the information display device. Although the power supplied by the solar charging panel 11 may gradually decrease with time, the power supplied from the solar charging panel 11 can effectively extend the operating duration of the information display device, thereby reducing the frequency of battery replacements (e.g., of the battery 13).

In order to extend the operating duration of the information display device, the control circuit 16 is configured to not operate continually, but instead operates periodically. In one embodiment, a timer within control circuit 16 will wake up control circuit 16 at regular intervals. After the control circuit 16 is woken up, the control circuit 16 immediately connects to a server or a gateway through a preset/default network connection setting, so as to obtain data corresponding to the information display device, and updates/refreshes the visual information displayed on the electronic paper 15. When the data displayed by the electronic paper 15 is refreshed, the control circuit 16 immediately enters into sleep mode and awaits the next wake up process.

In another embodiment, the control circuit 16 includes a first circuit and a second circuit. The second circuit includes a timer. The timer is configured to wake up the first circuit at a predetermined periodic time interval. Upon awaken, the first circuit is configured to wirelessly connect to a server or gateway through a predetermined network connection setting for receiving data therefrom. If the data indicates that the visual information on the electronic paper 15 needs to be updated, the first circuit enables the second circuit and simultaneously acquires data update from the server or the gateway, and the electronic paper 15 is updated by the second circuit. If the data indicates that the visual information on the electronic paper 15 requires no update, the first circuit immediately enters into sleep mode and waits for next wake up event.

In addition, in order to reduce the power consumption of the information display device, the design of the voltage regulating circuit 12 may also aim primarily toward reducing power consumption. In one embodiment, the voltage regulating circuit 12 includes only one Zener diode and two Schottky diodes to reduce the power consumption of the voltage regulating circuit 12. In another embodiment, the voltage regulating circuit 12 includes a boost circuit, a step-down (e.g., buck) circuit, a charging circuit, a protection circuit, and the like. However, it should be noted that the power consumed by these additional circuits cannot be greater than an initial value. For example, in an scenario where the control circuit 16 is woken up every 10 minutes, the information display device consumes 50 mW of power per refresh, and the solar charging panel 11 is capable of supplying 70 mW of energy every 10 minutes, then the initial value is selected to be 20 mW.

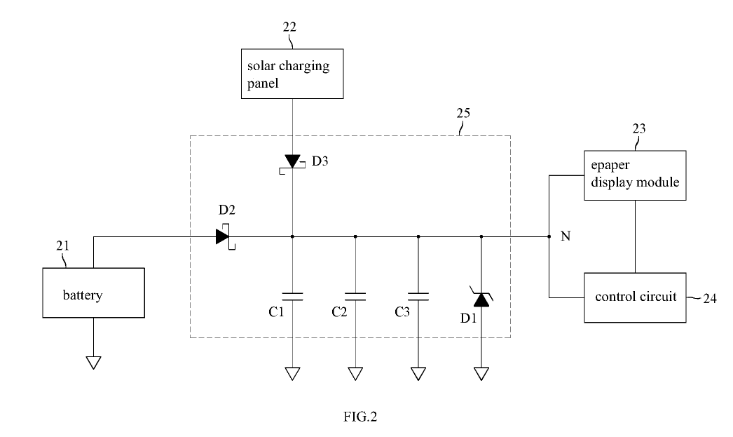

FIG. 2 is a functional block diagram of an embodiment of an information display device according to the present disclosure. The information display device includes a battery 21, a solar charging panel 22, an electronic paper display module 23, a control circuit 24, supercapacitors C1 to C3, and a voltage regulating circuit 25, wherein the voltage regulating circuit 25 includes only a Zener diode D1 and Schottky diodes D2 and D3. The control circuit 24 includes at least one controller and a wireless network module.

The negative electrode of the battery 21 is grounded and the positive electrode is coupled to the anode of the Schottky diode D2. The cathode of the Schottky diode D2 is coupled to node N. The solar charging panel 22 is coupled to the anode of the Schottky diode D3, and the cathode of the Schottky diode D3 is coupled to node N. The anode of the Zener diode D1 is grounded and its cathode is coupled to node N. The electronic paper display module 23 and the control circuit 24 are respectively coupled to node N, and operate to receive a voltage therefrom. The control circuit 24 is configured to transmit display data to the electronic paper display module 23. The control circuit 24 is configured to establish connection to a server through a wireless communication module (not shown) to obtain relevant information, and convert the relevant information to visual information for display. The supercapacitors C1-C3 each have a first terminal and a second terminal, where the second terminals are grounded, while the first terminals are coupled to node N.

In this embodiment, the operating voltage range of the electronic paper display module 23 is about 2.7V to 3.6V, and the operating voltage range of the control circuit 24 is about 2.1V to 3.8V. Accordingly, the voltage regulating circuit 25 is configured to maintain the voltage of the output terminal of the power supply in a range between 2.7V and 3.6V, so that the electronic paper display module 23 and the control circuit 24 can operate properly. In this embodiment, the upper limit of the tolerable voltage of the supercapacitors C1 to C3 is about 5.5V. It should be noted that, the upper tolerable voltage of the supercapacitors C1-C3 should be higher than the upper regulating range limit of the voltage regulating circuit 25 (e.g., 3.6V) to protect the supercapacitors C1-C3. In this embodiment, the clamping voltage of the Zener diode D1 is about 3.5V. Once the voltage at node N is higher than 3.5V, excess power would be discharged through the Zener diode D1 to ensure that the electronic paper display module 23 and the control circuit 24 do not receive excessive voltage. In short, the clamping voltage of the Zener diode D1 should be lower than the highest operating voltage of the electronic paper display module 23 and the control circuit 24.

When the battery 21 is electrically connected to the voltage regulating circuit 25, if the supercapacitors C1 to C3 are not yet fully charged, the output voltage of the battery 21 will charge the supercapacitors C1 to C3 with priority. At the same time, the solar charging panel 22 will continue to charge the supercapacitors C1 to C3. Once the voltage at node N is higher than the clamping voltage of the Zener diode D1, excess power will be discharged through the Zener diode D1.

For example, in one scenario, the initial voltage of the battery 21 is 4.2V, at which time the battery 21 charges the supercapacitors C1-C3. Because the voltage at node N is higher than the clamping voltage of the Zener diode D1 (e.g., 3.5V), the excess power will be discharged through the Zener diode D1. At the same time, the solar charging panel 22 will continue to charge the supercapacitors C1 to C3. When the voltage of the supercapacitors C1-C3 are slightly lower than the clamping voltage of the Zener diode D1 and slightly higher than the voltage of the battery 21, the power required by the electronic paper display module 23 and the control circuit 24 will be provided by supercapacitors C1-C3. When the electronic paper display module 23 is operated, if the instantaneous voltage of the supercapacitor C1-C3 (e.g., 3.1V) is lower than the voltage of the battery 21 (e.g., 3.2V), the battery 21 will provide power to the supercapacitor C1-C3, the control circuit 24, and electronic paper display module 23. When the solar charging board 22 continues to charge the supercapacitors C1-C3, so that the voltage of the supercapacitors C1-C3 becomes higher than the voltage of the battery 21, the supercapacitors C1-C3 will be the primary power supplier for the control circuit 24 and the electronic paper display module 23 in next cycle.

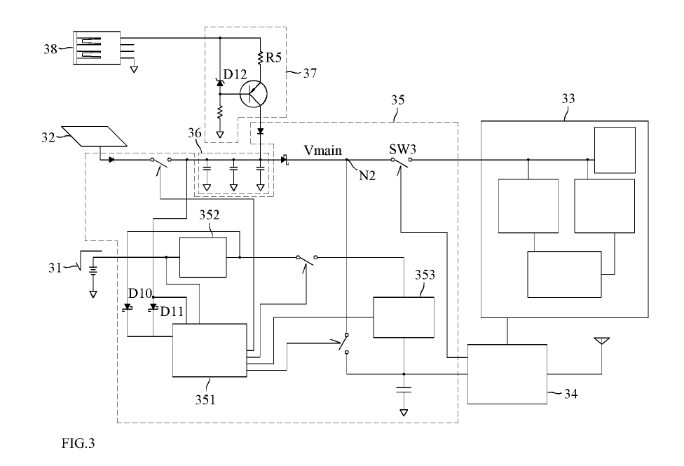

FIG. 3 is a functional block diagram of another embodiment of the information display device according to the present disclosure. The exemplary information display device includes a battery 31, a solar charging panel 32, an electronic paper display module 33, a control circuit 34, a voltage regulating circuit 35, a supercapacitor module 36, a constant current circuit 37, and a USB connector 38. In this embodiment, among the four pins in the USB connector 38, a VCC pin is connected to the constant current circuit 37, and the GND pin is thereof is connected to the system ground potential. The D+ and D− pins are not connected. In other words, the USB connector 38 is not connected for data transfer. In the present embodiment, the supercapacitor module 36 of the information display device will be charged by external power only when being connected to an external power source through the USB connector 38. The USB connector is arranged is such a way that, the user cannot obtain data from the information display device or write data into the information display device through the USB connector 38. In another embodiment, the constant current circuit 37, the USB connector 38, and the diode D9 may be omitted.

The voltage regulating circuit 35 is configured to control and maintain the voltage on the main line (the voltage at node N2) within operable voltage range of the electronic paper display module 33 and the control circuit 34. In one embodiment, the operating voltage of the control circuit 34 ranges from 2.5V to 3.8V, and the operating voltage range of the electronic paper display module 33 ranges from 2.7V to 5.5V. The voltage regulating circuit 35 controls the voltage on the main line and maintained it at about 2.8V-5.3V.

The voltage regulating circuit 35 includes a diode D2 coupled between the solar charging board 32 and the switching device SW2. The other end of the switching device SW2 is coupled to the anode of the supercapacitor module 36 and the Schottky diode D5 through node N1. The cathode of the Schottky diode D5 is coupled to the switching device SW3 through node N2, and the other end of the switching device SW3 is coupled to the electronic paper display module 33.

The voltage regulating circuit 35 further includes a microcontroller 351, a boosting circuit 352, and a step-down circuit 353. The microcontroller 351 is coupled to the battery 31, to node N1 through the Schottky diode D11, and to the output of the booster circuit 352 via the Schottky diode D10. The microcontroller 351 further controls whether the switching device SW4 and the switching device SW5 are turned on or off. When the microcontroller 351 finds that the voltage of node N1 is lower than a critical value (e.g., 2.7V-2.8V) the microcontroller 351 turns on the switching devices SW2 and SW4. At which time the solar charging board 32 charges the supercapacitor module 36. The electronic paper display module 33 and the control circuit 34 are thus powered by the battery 31.

When the microcontroller 351 detects that the voltage of node N1 is lower than a predetermined value (e.g., 3.8V), the microcontroller 351 turns on the switching device SW5. When the switching device SW5 is turned on, the capacitor C11 may provide the large instantaneous current required to turn on the control circuit 34, thus preventing an excessive current draw from node N2 due to the switch on of the control circuit 34. If the capacitor CII is not provided to offer the large instantaneous current required for the control circuit 34, once the current in the supercapacitor module 36 is depleted, the control circuit 34 will be on battery power (e.g., from battery 31). As a result, the operational duration of the battery 31 will be reduced. When the microcontroller 351 detects that the voltage at node N1 is within a predetermined voltage range (e.g., 3.8V-4V), the microcontroller 351 turns off the switching device SW5.

Generally speaking, as long as the solar charging panel 32 charges the supercapacitor module 36 for a sufficient time, the undesirable situation where the supercapacitor module 36 being incapable of providing startup current for the control circuit 34 will not occur (particularly in the instant example, where the electronic paper display module 33 is periodically refreshed, and the refresh period is greater than the aforementioned charging time). In some embodiments, the information display device of the instant embodiment is provided with an external update button configured to instantly refresh the display information upon manual request. However, if the time of the external update is too close to the time of the automatic update, the condition where the supercapacitor module 36 being unable to provide the required turn-on current for the control circuit 34 may occur. Accordingly, in the present embodiment, it is necessary to use the capacitor C11 to provide an large instantaneous start-up current for turning on the control circuit 34. In the present embodiment, the capacitor C11 is charged by the battery 31 or the supercapacitor module 36 through the step-down circuit 353.

If the supply voltage of the battery 31 is greater than the operating voltage of the electronic paper display module 33 or the control circuit 34, the microcontroller 351 sends an enable signal to enable the voltage reduction circuit 353 to adjust the output voltage of the battery to the electronic and maintain it within the proper operation voltage range of the epapaer module 33 and the control circuit 34. In other words, if the supply voltage of the battery 31 is not greater than the operating voltage of the electronic paper display module 33 or the control circuit 34, the step-down circuit 353 will not operate (and only the input voltage will be allowed to bypass to the output). In the present embodiment, the buck circuit 353 can accept an input voltage range from about 3.8V to 5V, and the buck circuit 353 has an output voltage of 3.3V.

In another embodiment, the buck circuit 353 consists of passive components, which can set a maximum output voltage. Once the input voltage is greater than the set maximum output voltage, the buck circuit 353 is configured to directly step down the input voltage to the maximum output voltage.

In this embodiment, the microcontroller 351 is continuously powered by the battery 31 or the supercapacitor module 36, and the function of the microcontroller 351 is to perform power allocation for the voltage value of the supercapacitor, so that the operating duration for the battery 31 can be increased.

In one embodiment, the control circuit 34 includes a control unit and a wireless module (not shown). The control circuit 34 does not operate continuously, but rather periodically. In an embodiment, a timer in the control unit is provided to wake up the control unit at regular intervals. The control unit then subsequently connects to a server or gateway according to a preset network connection setting through the wireless module. Upon obtaining the data associated with the visual information for the display device, the control unit turns on the switch device SW3 to enable the electronic paper display module 33. Then, the control unit transmits the received data to the electronic paper display module 33 for refreshing the display content thereon through the control bus. After the electronic paper display module 33 refreshes the display content thereof, the control unit turns off the switch device SW3. Subsequently, the control unit and the wireless module immediately enter into sleep mode, awaiting next wake up event.

In another embodiment, if a specific bit in the data acquired by the control unit is of a specific value, such as a logic 0, it indicates that the display content of the electronic paper display module 33 need not be changed. Accordingly, the control unit would provide the received data into the storage of the epaper module 33 without requesting it to refresh the displayed content, thereby conserving energy usage. In other words, in the present case, the electronic paper display module 33 is configured to perform two operations: writing the display data (e.g., externally received data) into the storage device, and updating the display screen based on the display data in the storage device. Therefore, if the display material received by the electronic paper display module 33 is the same as that previously displayed on the screen, the electronic paper display module 33 will not perform screen refresh operation.

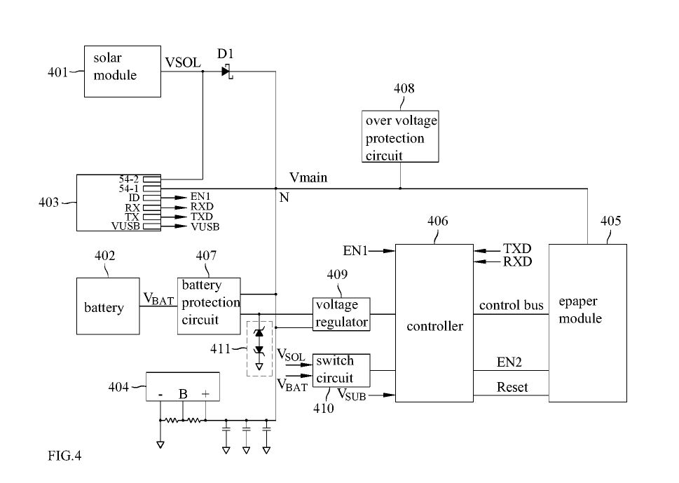

FIG. 4 is a functional block diagram of another embodiment of the information display device according to the present disclosure. The information display device includes a solar module 401, a battery 402, a connector 403, a supercapacitor 404, an electronic paper (epaper) module 405, a controller 406, a battery protection circuit 407, an overvoltage protection circuit 408, a voltage regulator 409, a switch circuit 410, and an antistatic surge circuit 411. In this embodiment, the controller 406 is integrated with a wireless network module. In the present embodiment, the battery protection circuit 407, the overvoltage protection circuit 408, the voltage regulator 409, the switch circuit 410, and the antistatic surge circuit 411 form a voltage adjustment circuit as previously described. In this embodiment, the solar module 401 includes a solar panel and an supercapacitor (not shown) for storing power and supplying power when the back end circuit requires a large current. In addition, as described above, during the sleep mode of the information display device, the solar panel in the solar module 401 can continue to charge the internal supercapacitor to provide sufficient current to wake up the information display device.

When the output voltage V_{SOL} of the solar module 401 is lower than the voltage Vmain (voltage on the main line)on the main line, the Schottky diode D1 will not output the output voltage V_{SOL} to the main line, so the solar module 401 will charge the internal supercapacitor until the V_{SOL} is equal to the voltage Vmain. Once the other components on the main line require power and the voltage Vmain (voltage on the main line) on the main line is less than the output voltage V_{SOL}, the supercapacitor 404 and the supercapacitor inside the solar module 401 will act to provide power.

The connector 403 has a plurality of pins. In one embodiment, an USB connector can be used, but the pin connection arrangement would be different from that used in a normal USB pin. The connector 403 has six pins, which are labeled 5V_1, 5V_2, ID, RX, TX, and VUSB, respectively. Pins 5V_1 and 5V_2 provide 5V and are coupled to the cathode and anode of the Schottky diode D1, respectively. Therefore, once the connector 403 is properly connected to an external device, the solar module 401 would not output a voltage to the main line (e.g., node N). In another embodiment, the pins 5V_1 and 5V_2 can be combined into a single pin.

When an external cable is connected to the connector 403, the pin VUSB transmits a signal V_{USB} to the controller 406, informing the controller 406 the presence of an external device. If the external device needs to communicate with the information display device, data can be transmitted to or received from the controller 406 via the pins RX and TX. In an embodiment, the information display device can perform a firmware update via the network. When the controller 406 receives a firmware update indication from the server, the controller 406 may wake up and perform a firmware update at a specified time. It should be noted that the controller 406 does not update the display material of the electronic paper module 405 when updating firmware. In another embodiment, when the secondary controller 406 is woken up, the controller 406 would not request the server to obtain updated data for the electronic paper module 405 when the controller 406 receives the firmware update instruction last time.

In another embodiment, the information display device can be connected to the connector 403 through an external device for firmware update. When the external device is to perform the firmware update, the pin ID will issue an enabling signal EN1 to the controller 406, informing the controller 406 that a firmware update is to be performed. In another embodiment, the pin ID will be pulled up to a high voltage level to inform the controller 406 to perform a firmware update. The controller 406 communicates with the external device via the TX and RX pins of the connector 403 and performs a firmware update.

In another embodiment, the server connected to the information display device can perform firmware update through remote control. When the information display device is awakened, the information display device first obtains a data, e.g., control command and the display data, from the server (or gateway) through default network connection information. The operation of updating the electronic paper module 405 can be referred to previous descriptions.

When the server needs remote update, the time of the firmware update is added to the control command (for example, at 12:00 AM), so when the information display device is woken up at the predetermined time, the controller 406 would not perform data refresh for the electronic paper module 405, but only the firmware update process. When the firmware update procedure is performed, the controller 406 downloads a new firmware from the server to a storage space of the information display device, and performs a firmware update. In an embodiment, after the firmware is updated, the information display device is restarted, and the controller 406 reports to the server that firmware update is completed. Subsequently, the information display device enters into sleep mode to wait for next wake up event. It should be noted that, the storage space for firmware of the epaper module 405 may be different from that for the display data.

In another embodiment, when the server needs remote update, the time of and source location of the firmware files may be added to the control instructions (may be directed to another server with a different location or URL). When the information display device is woken up at a predetermined time, the controller 406 would not update the display material of the electronic paper module 405, but only performs the process of firmware update. When the firmware update procedure is in progress, the controller 406 will connect to a specific location to obtain a new firmware files and perform firmware update. In one embodiment, after the firmware is updated, the information display device is restarted and the controller 406 reports to the server that firmware update is completed. Subsequently, the information display device enters into sleep mode and waits for next wake up event. It should be noted that, the storage space for firmware of the epaper module 405 may be different from that for the display data.

The battery 402 is coupled to node N (e.g., a first node) through the battery protection circuit 407. The battery protection circuit 407 is used to protect the battery 402 from being over-drained and lose operation life. Moreover, in the absence of the battery protection circuit 407, once the voltage on the main line is lower than the voltage of the battery 402, the battery 402 will quickly charge the supercapacitor 404 through the battery protection circuit 407. In one embodiment, the protection circuit 407 is configured to limit the output current of the battery 402. In another embodiment, the protection circuit 407 only turns on when the voltage Vmain (voltage on the main line)on the main line is detected to be lower than a predetermined voltage (e.g., 3.2V), so as to allow the battery 402 to charge the supercapacitor 404. In another embodiment, the protection circuit 407 is only turned on when Vmain (voltage on the main line)on the main line is detected to be lower than a predetermined value (e.g., 3.2V) and when the controller 406 is not awaken, so as to allow the battery 402 to charge the supercapacitor 404.

The voltage regulator 409 is grounded through an anti-static surge circuit 411 and coupled to the battery protection circuit 407 and node N. The antistatic surge circuit 411 is used to prevent surge voltage/current, so as to protect the regulator 409 and the battery protection circuit 407. Voltage regulator 409 is arranged to receive voltage Vmain (voltage on the main line)from node N or voltage from the battery 402, and configured to provide voltage to the controller 406. In one embodiment, the voltage regulator 409 will be turned on to enable the controller 406 at a particular time or when a particular wake-up signal is received.

In addition, the overvoltage protection circuit 408 is coupled to node N to prevent damage to the electronic paper module 405 and the controller 406 from excessive voltage thereform.

The controller 406 receives voltage Vmain (voltage on the main line)through the voltage regulator 409, and receives voltage V_{SOL} from the solar module 401 and the voltage VBAT from the battery 402 through the switch circuit 410. The controller 406 can transmit information of the voltage values to the server, so as to allow back-end personnel to monitor the status of the current information display device (or to determine whether the battery 402 needs to be replaced). In general, the switch circuit 410 is turned off. Only when the controller 406 receives a request the switch circuit 410 would be turned on. Upon the measurement and return of the voltage value, the switch circuit 410 is turned off again.

The controller 406 is configured to transmit the display data to the electronic paper module 405 through the bus. Generally, the controller 406 sends a request to the electronic paper module 405 through the pin EN2 for requesting relevant parameter information thereof, such as model, size, resolution, and the like. If the electronic paper module 405 does not reply the parameter request for a period of time (e.g., in 5 to 10 seconds), the controller 406 will send the request to the electronic paper module 405 again or directly send a reset signal Reset to the electronic paper module 405, so as to cause reset of the electronic paper module 405. At this time, the screen of the electronic paper module 405 will display a factory default screen or a preset screen, and awaits the controller 406 to transmit the new display data/material.

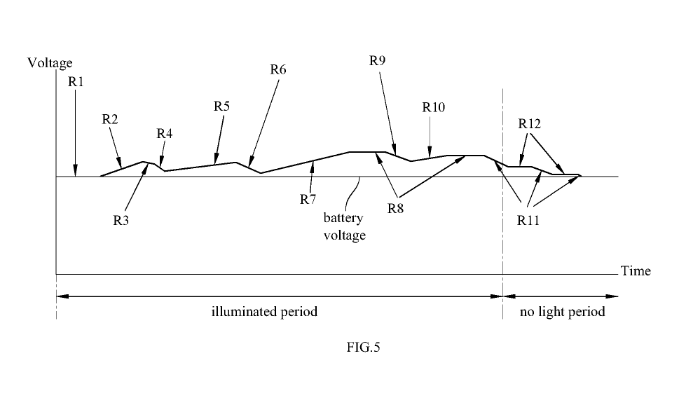

Referring to FIG. 5, which shows an exemplary operational waveform diagram that indicates the voltage change of the battery power supply and the voltage of the output power supply at different stages of operation. The diagram is separated into a luminescent period and a no-light period, denoted by a first area having receipt of light input and a second area without light input. Specifically, the point R1 represents the start of using battery power; point R2 indicates that solar power source starts charging the supercapacitor to increase voltage; while points R3, R4, R6, R9, and Ru illustrate voltage load that draw power output and cause a drop of voltage level. segment R4 shows a drop in voltage value to that of the battery voltage level due to continuous drawing of output power under an insufficient power condition; segments R5, R7, and R10 illustrates the charging of the supercapacitor from the solar power source, so as to cause the rise of voltage level; segment R8 shows a charging to a saturation state where voltage no longer rises; and point R12 shows that the voltage level no longer rises due to the absence of light energy input.

Therefore, the priority of the input power is to use USB power, solar power, and then battery power. That is, when the power of the USB power source is sufficient, the USB power is preferentially used because the USB power is generally converted from household power (or devices storing a large amount of energy). If the power of the USB power source is insufficient but a solar power source is available/sufficient, the solar power source is used instead of the battery power source (even if battery power level is high) because battery power is relatively scarce/precious.

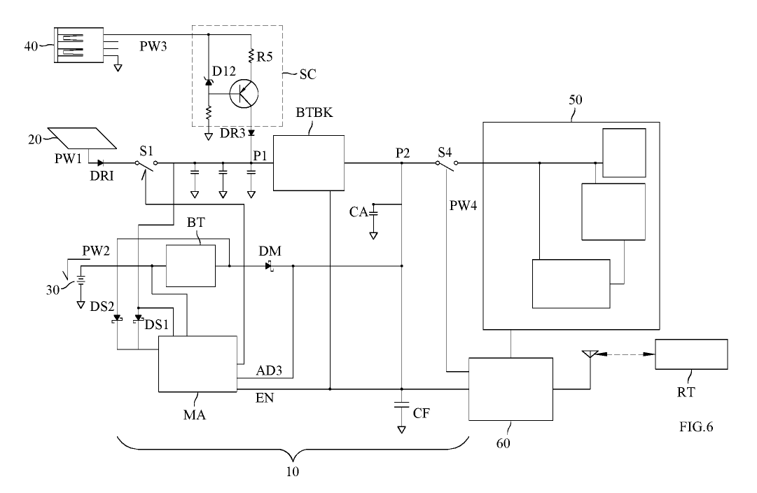

FIG. 6 shows a schematic diagram of an exemplary information display device similar to that shown in FIG. 4. Accordingly, comparable features and components will not be described again for the brevity of disclosure. In this exemplary information display device, the voltage regulator 10 includes an auxiliary controller MA, a first rectifying diode DR1, a third rectifying diode DR3, a first switch S1, a fourth switch S4, a boosting unit BT, a step-up and step-down unit BTBK, a current stabilizing unit SC, a filter capacitor CF, and at least one supercapacitor C1, C2.

The first rectifying diode DR1 and the first switch S1 are connected in series between the solar power source PW1 and the first node P1. The first rectifying diode DR1 is configured to receive power from the solar power source PW1. The first switch S1 is also connected to the first node P1.

The boosting unit BT and the intermediate Schottky diode DM are connected in series between the battery power source PW2 and the second node P2. The boosting unit BT is arranged to receive the power from battery power PW2, and when battery power PW2 reaches below a threshold value (e.g., 3V), to output voltage to the intermediate Schottky diode DM to boost voltage level (e.g., back to 3V). In addition, the boosting power of the boosting unit BT is provided to the auxiliary controller MA via the first Schottky diode DS1. The first Schottky diode DS1 is further connected to the first node P1 via the second Schottky diode DS2.

The current stabilizing unit SC and the third rectifying diode DR3 are connected in series between the USB power source PW3 and the first node P1, wherein the steady current unit SC receives and stabilizes the USB power source PW3, and transmits the stabilized power output to the first node P1 through the third rectification diode DR3.

The supercapacitors C1 and C2 are connected to the first node P1 for receiving power from the solar power source PW1, the battery power source PW2, and the USB power source PW3 for charging.

The first node P1 is connected to the second node P2 via the boost buck unit BTBK. The output voltage of the boost buck unit BTBK is, for example, 3V to 3.6V. The second node P2 is connected to the output power source PW4 via the fourth switch S4. In addition, an auxiliary supercapacitor CA is disposed between the second node P2 and the ground to stabilize the voltage at the second node P2. The filter capacitor CF is connected to the second node P2 for filtering the voltage of the second node P2.

The auxiliary controller MA is configured to receive power from the first Schottky diode DS1 or the second Schottky diode DS2, and in particular, to detect the voltage of at first node P1 and the voltage of the battery power source PW2, and accordingly generate and transmit an enable signal EN to the step-up and step-down unit BTBK and the control circuit unit 60, thereby controlling the step-up and step-down unit BTBK and the control circuit unit 60.

In addition, the control circuit unit 60 is configured to receive voltage from the second node P2, and upon receipt of the enable signal EN from the auxiliary controller MA, to wirelessly receive patient information from the server unit RT. And accordingly, the control circuit unit 60 turns on the fourth switch S4 and allow data transmission to the epaper module 50 for display; and after a preset time interval, turns off the fourth switch S4 to achieve power saving.

Therefore, the auxiliary controller MA can control the power supply priority/sequence of the input power source (through controlling the first switch S1) as follows: the USB power source PW3, the solar power source PW1, and the battery power source PW2. That is, the USB power source PW3 is of the highest priority.

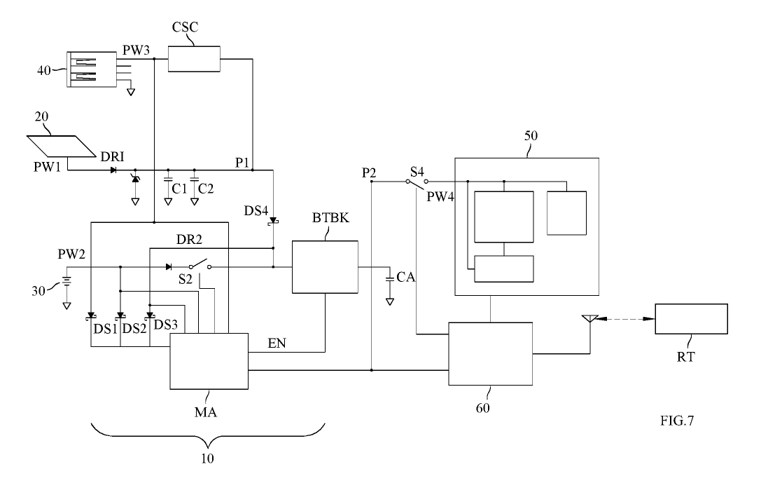

FIG. 7 shows a schematic diagram of an exemplary information display device that is similar to that shown in FIG. 6. Accordingly, comparable features and components will not be described again for the brevity of disclosure. The voltage regulator 10 of the device includes an auxiliary controller MA, a first rectifying diode DR1, a second rectifying diode DR2, a second switch S2, a fourth switch S4, a step-up and step-down unit BTBK, a current source CSC, and at least a supercapacitor C1, C2. The first rectifying diode DR1 is connected in series between the solar power source PW1 and the first node P1. The first rectifying diode DR1 is configured to receive power from the solar power source PW1, and is further configured to connect the first node P1. The second rectifying diode DR2 and the second switch S2 are connected in series between the battery power source PW2 and the first node P1, wherein the second rectifying diode DR2 receives power from the battery power PW2. The second switch S2 is connected to the first node P1. Further, the boost buck unit (step up/down) BTBK is connected between the first node P1 and the second node P2.

The USB power source PW3 is connected to the first node P1 via the current source CSC, and is connected to the auxiliary controller MA via the first Schottky diode DS1. The supercapacitors C1 and C2 are connected to the first node P1 for receiving power from the USB power source PW3 and the solar power source PW1 for charging, and can receive the battery power source PW2 via the second rectifying diode DR2 and the second switch S2 (for charging).

Further, the battery power source PW2 is connected to the auxiliary controller MA via the second Schottky diode DS2, and the second Schottky diode DS2 is further connected to the boost buck unit BTBK via the third Schottky diode DS3. Furthermore, the fourth Schottky diode DS4 is connected between the first node P1 and the boost buck unit BTBK, and the second node P2 is connected to the output power PW4 via the fourth switch S4. The auxiliary super capacitor CA is provided between the second node P2 and the ground for stabilizing the voltage at the second node P2.

The auxiliary controller MA is configured to receive power from the first Schottky diode DS1, the second Schottky diode DS2, or the third Schottky diode DS3. The auxiliary controller MA generates and transmits an enable signal EN to the boost buck unit BTBK by detecting the voltage of the first node P1, the voltage of the battery power PW2, and the voltage of the second node P2, thereby controlling the operation of the boost buck unit BTBK.

Furthermore, the control circuit unit 60 operates by receiving the voltage of the second node P2. Upon receiving patient information from server RT through wireless connection, the control circuit unit 60 turns on the fourth switch S4 to enable data transmission to the epaper module 50 for display, and turns off the fourth switch S4 after a preset period of time. The auxiliary controller MA turns on the second switch S2 when the voltage at the first node P1 is lower than a preset value, so as to use the batter power source PW2 as input power supplier, and further generate voltage at the second node P2 via the boost buck unit BTBK.

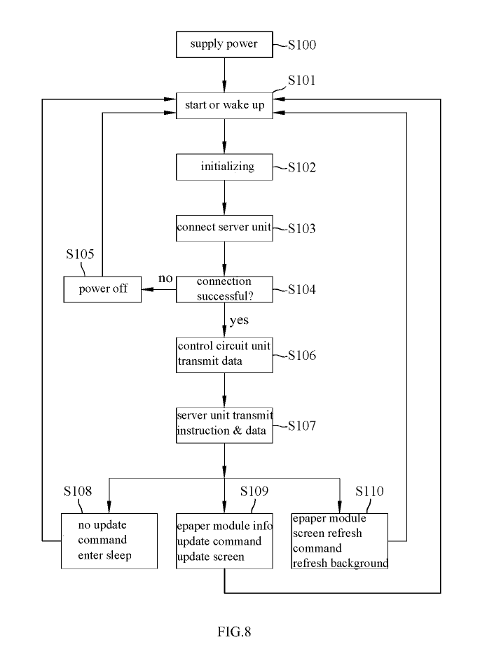

FIG. 8 shows an operation flow diagram of the control circuit unit of an information display device in accordance with one embodiment of the present disclosure. The operation of the control circuit unit comprises:

process S100: receiving normal power supply;

process S101: initializing or waking a control circuit unit;

process S103: connecting to a server unit;

process S104: determining if connection is successful; if within time-out limit, continue connection attempt;

if connection is not successful and time is out, proceed to process S105, in which epaper module and the control circuit are turned off and enters sleep mode, than return to S101;

if the connection is successful, proceed to S106, in which the control circuit transmits information of the epaper module to the server unit;

process S107: the server unit transmits instruction and data, wherein the instruction does not include update command, epaper module update command, and screen refreshing command;

process S108: when instruction from server unit contains no update command, power off the epaper module and let control circuit enter into sleep mode, then return to S101;

process S109: when instruction from server unit contains update command, wait until epaper module to enter standby mode, and upon the latest update, refresh a background image of the epaper module, and update information of the epaper module when it is not the last update (i.e., background image data update is stored in internal memory device), and return to S103; and

process S110: when instruction contains epaper module screen refresh command, the control circuit refreshes the display content of the epaper module, and subsequently powers off the epaper module enters into sleep mode, then return to process S101.

The wake-up triggering factors of the above-mentioned electronic paper modules may include: RTC wakeup (i.e., fixed wake-up); and hardware GPIO pin change wakeup (user manual wakeup).

Further, data transmission between the control circuit unit and the server unit can incorporate encryption protection technology to increase communication security. For example, before receiving the information, the CRC or Check Sum mechanism may be applied to ensure the correctness of the data transmission. When the control circuit unit transmits data to the server unit, the operating status, time, battery power, ambient temperature and humidity information may also be included.

The updated content transmitted from the server unit to the electronic paper module can be transmitted in whole or through multiple partial transmissions. That is, by transmitting only a portion of a data file required update the display device, transmission volume and time may be reduced, thereby compensating for the limited build-in memory in the display device while achieving the purpose of power saving.

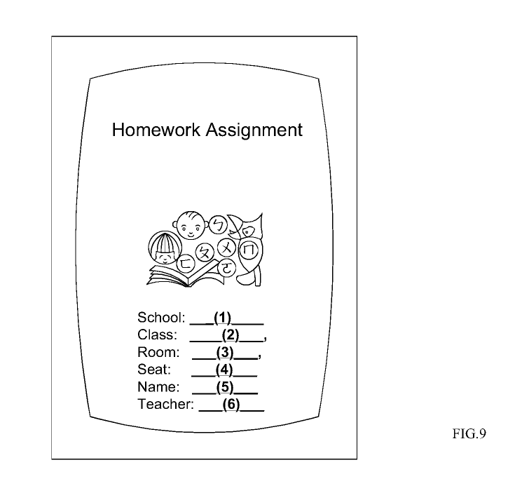

For example, in the exemplary operational example of FIG. 9, the image content on the information display device may be divided into six partial regions 1, 2, 3, 4, 5, 6. During image updating process, the whole page may be updated, including the background (which requires higher power consumption). Alternatively, the update process may refresh only a certain partial area, such as the partial area of label 5, or a plurality of partial areas, such as the regional areas of labeled by 1, 3, and 5 (so as to achieve better power saving).

It should be noted that, the circuit components or the operation modes of the exemplary ringer control device described in the foregoing embodiments may be interchanged or combined, given no substantial conflicts in compatibility. Thus, the scope of disclosure should not be limited to the specific embodiments described herein.

The embodiments shown and described above are only examples. Many details are often found in this field of art thus many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. An information display device adaptable in a ward to display patient information, comprising:
a control circuit;
an electronic paper (epaper) module configured to be controlled by the control circuit for displaying patient information;
a solar charging panel configured to output a first voltage;
a battery configured to output a second voltage;
a rechargeable energy storage device, and
a voltage regulating circuit coupled to the solar charging panel, the battery, and the rechargeable energy storage device and configured to provide an operational voltage to the control circuit and the epaper module.

2. The device of claim 1, wherein
the operational voltage includes a lower limit value and an upper limit value;
the lower limit value is higher than the lowest operating voltage of either one of the control circuit and the epaper module; and
the upper limit value is lower than highest operating voltage of either one of the control circuit and the epaper module.

3. The device of claim 1, wherein when the battery is electrically connected to the voltage regulating circuit,
when the voltage regulating circuit detects that a voltage of the battery is higher than an operating voltage on a main line, the voltage regulating circuit enables the battery to charge the rechargeable energy storage device; and
when the rechargeable energy storage device is fully charged, and the voltage of the battery is still higher than the operating voltage on the main line, the voltage regulating circuit discharges the battery until the voltage of the battery substantially equals to the operating voltage of the battery.

4. The device of claim 1, wherein
the control circuit is configured to be operable in a working mode and a sleep mode; and
upon awaken, the control circuit is configured to wirelessly connect to a server through a predetermined network connection setting, obtain a display data, cause a display refresh on the epaper module, and enter sleep mode after the display refresh.

5. The device of claim 1, wherein the rechargeable energy storage device includes one or more supercapacitor.

6. The device of claim 1,
wherein the control circuit includes a first circuit and a second circuit;
wherein the second circuit includes a timer, wherein the timer is configured to wake up the first circuit at a predetermined periodic time interval,
wherein the first circuit is configured to wirelessly connect to a server through a predetermined network connection setting for receiving a data.

7. The device of claim 6, wherein if the data indicates no need for display refresh, the first circuit enters into sleep mode promptly.

8. The device of claim 1, wherein the voltage regulating circuit consists of:
a first Schottky diode, the anode thereof being connected to the positive terminal of the battery, the cathode thereof being connected to a first node;
a second Schottky diode, the cathode thereof being connected to the node, and the anode thereof being connected to the solar charging panel; and
a Zener diode, the cathode thereof being connected to the first node, and the anode thereof being connected to ground,
wherein the rechargeable energy storage device is coupled the first node.

9. An information display device, comprising:
a control circuit;
an electronic paper (epaper) module, configured to be controlled by the control circuit for displaying patient information;
a USB connector, wherein only a VCC pin thereof is coupled to a constant current circuit, and a GND pin thereof is coupled to ground;
a solar charging panel;
a battery;
a rechargeable energy storage device; and
a voltage regulating circuit coupled to the solar charging panel, the constant current circuit, the battery, and the rechargeable energy storage device, and configured to provide the control circuit and the epaper module an operational voltage,
wherein the constant current circuit is configured to provide power to the voltage regulating circuit only when the USB connector is coupled to an external device.

10. An information display device adaptable in a ward to display patient information, comprising:
a solar module;
a battery;
a supercapacitor module;
a voltage regulating circuit coupled to the solar module, the batter, and the supercapacitor module;
an electronic paper (epaper) module; and
a controller, configured to control the epaper module for displaying patient information, wherein the voltage regulating circuit selectively charges the supercapacitor module using the solar module and the battery, wherein the epaper module and the controller are primarily powered by the supercapacitor module.

11. The device of claim 10, wherein the solar module further comprises a solar panel and a second supercapacitor, wherein when the controller and the epaper module are in sleep mode, the second supercapacitor is configured to store energy generated by the solar panel, and upon awaken of the controller, to provide power thereto.

12. The device of claim 10, further comprises a Schottky diode coupled between the solar panel and a first node, wherein when output voltage of the solar module is lower the a voltage at the first node, the Schottky diode prevents voltage output to the first node, and the solar panel charges the second supercapacitor until output voltage thereof equals to the voltage at the first node.

13. The device of claim 10, wherein the voltage regulating circuit further comprises a protection circuit coupled between the battery and the first node configured to limit current output of the battery.

14. The device of claim 13, wherein the battery protection circuit is only turned on when the voltage at the first node is below a predetermined value and the controller is in sleep mode.

15. The device of claim 10, wherein the voltage regulating circuit further comprises:
a voltage regulator configured to provide an operating voltage for the controller; and
an anti-static surge circuit coupled between the voltage regulator and ground for protecting the voltage regulator.

16. The device of claim 10, wherein the controller is configured to send a request to the epaper module for obtaining a parameter thereof, and if the epaper module does not return the parameter within a predetermined time, the controller resets the epaper module.
